(19) United States
(12) Patent Application Publication
(10) Pub. No.: US 2006/0122576 A1
(43) Pub. Date: Jun. 8, 2006

(54) Fluid delivery system for use with a surgical pumping unit

(76) Inventors: Khalid Raja, Grafton, MA (US); Frank D'Amelio, Los Olivos, CA (US); Dennis Caudle, Solvang, CA (US); Roger Raetzman, Kenosha, WI (US); Kevin Wood, Compoc, CA (US); Marvin Parrett, Racine, WI (US)

Correspondence Address: GANZ LAW, P.C., P O BOX 2200, HILLSBORO, OR, 97123, US

(21) Appl. No.: 10/692,363

(22) Filed: Oct. 22, 2003

(51) Int. Cl. A61K 9/22

(52) U.S. Cl. 604/890.1; 604/80; 604/250; 604/249

(57) ABSTRACT

A surgical irrigation system is provided in which a novel system of connecting and/or supporting a pump to a fluid source reduces the possibility that the pump will become disconnected from the fluid source by requiring that the pump be supported by a support to establish fluid connection.

This invention claims the benefit of co-pending U.S. Provisional Application No. 60/291,583, entitled Fluid Delivery System For Use With A Surgical Pumping Unit, filed May 16, 2001, the entire disclosure of which is incorporated by reference as if set forth in its entirety for all purposes.

### BACKGROUND OF THE INVENTION

This invention relates to a system for providing fluid to a surgical site inside a body and in particular this invention relates to a connection system for connecting a pump or other device to a fluid source to supply fluid to the surgical site.

The use of surgical irrigation systems is known in the art. Such systems typically comprise an irrigation liquid source and a handpiece which has an inlet port connected to the irrigation liquid source, and an outlet port connected to, for example, a probe extending to an operative site within a body. Typically, a plastic bag containing irrigation fluid is suspended from an IV pole and is connected to the handpiece through a pumping device connected to the fluid bag through a tubular spike.

One example of such a system is disclosed in U.S. Pat. No. 5,484,402, which issued to Saravia et al. on Jan. 16, 1996, the disclosure of which is hereby incorporated by reference for all purposes. Saravia et al. disclose a surgical irrigation system that includes a self-contained pumping unit remotely located from the handpiece to pump irrigation liquid to the handpiece, into the protruding hollow tip, and to the surgical site. The remotely located pumping unit comprises a power supply (i.e., a battery pack), which is activated by an electrical switch in the handpiece. Therefore, the self-contained pumping unit must be connected to the switch in the handpiece by running cumbersome electrical cable along the flexible tubing that connects the pumping unit to the electrical switch on the handpiece. Additionally, the pumping unit is directly connected to the fluid bag by a tubular spike connector that is connected to a luer-type connector on the fluid bag. The pumping unit may be suspended from the bag simply by the interconnection of the respective connectors or may be supported by a bracket. This system is limited in its arrangement since the pumping unit must be directly connected to the fluid bag. This creates a potentially hazardous situation. For example, some surgical rooms have limited space available for surgeons and other hospital personnel to move about. This increases the chance that the pumping unit might be bumped or knocked from its connection with the fluid bag resulting in a dangerous interruption in the delivery of fluid to a patient and creating unsanitary and/or unsafe operating room conditions. This is particularly true when the pumping unit is suspended from the fluid bag and not held in the bracket.

Another system is disclosed in U.S. Pat. No. 6,176,847, issued to Humphreys, Jr. et al. on Jan. 23, 2001, the disclosure of which is hereby incorporated by reference for all purposes. Humphreys, Jr. et al. disclose a surgical irrigation system that includes a fluid flow sensor device including a fluid accelerator to increase fluid flow to a surgical handpiece. The flow sensor device is directly connected to and supported from a fluid bag but may be adjustably fixed and supported on a vertical IV pole. While this system has proven effective, the present invention provides improvements in the construction and arrangement of irrigation system components.

### SUMMARY OF THE INVENTION

The present invention overcomes the problems described in the prior art surgical irrigation systems by providing novel systems and methods of connecting the pump or other fluid accelerating device to the fluid source.

The present invention provides a surgical irrigation system in which the pump must be supported in order to maintain its fluid connection with the fluid source. The system includes novel systems of connecting the pump to the fluid bag that reduces the chance that the pump will become disconnected from the fluid bag by requiring that the pump be supported and not suspended from the fluid bag. The present invention further allows the pump to be located a various positions so that it can be moved out of the way of surgeons or other hospital personnel further reducing the chance that the pump will be knocked from its connection to the fluid bag.

The present invention further provides means for automatically discontinuing fluid flow delivery if the pump is not properly supported for use.

In one embodiment the pump is connected to a fluid bag by at least one flexible tube. The flexible tube may be connected to the pump by a variety of connectors and/or fittings to accommodate a variety of arrangements. Plural flexible tubes may be connected to the pump for connection to one or more fluid bags.

In another embodiment the pump includes a switch that completes an electrical circuit to supply power to the pump motor only when the pump is located and/or supported by a supported. If the pump is not located in the support switch remains open and no power is supplied to the pump motor and no fluid will flow to the handpiece.

In another embodiment the pump is connected to the fluid bag by a connector that includes a valve that closes and prevents fluid flow if the pump is not supported in a support.

In yet another embodiment the pump is connected to the fluid bag by a connector that is supported by a bracket. The pump is constructed and arranged so that it cannot be connected to the fluid bag other than through the connector which itself is supported.

In still another embodiment the pump is connected to the fluid bag by a connector in which fluid flow is blocked until a user activates a release mechanism to start fluid flow.

These and other embodiments are described in more detail in the following detailed description and the figures.

### BRIEF DESCRIPTION OF THE DRAWINGS

FIG. 1 is a schematic drawing of a representative surgical irrigation system of the present invention.

FIG. 2 is a schematic cross-section of a preferred pump for use with the present invention.

FIG. 3 is a schematic diagram of a “Hall-Effect” switching circuit which is an embodiment of this invention and can be utilized in the pump of FIGS. 1 and 2.

FIG. 4 is a schematic drawing of the connection of a flexible tube to the pump impeller housing.

FIG. 5 is a schematic drawing of another embodiment of the system of FIG. 1.

FIG. 6 is a schematic drawing of a modified pump impeller housing.

FIG. 7 is a partial exploded schematic drawing of the flexible tube and its connection to the impeller housing.

FIG. 8 is a schematic drawing of another embodiment showing the pump suspended from an IV pole.

FIG. 9 is a partial schematic drawing showing another embodiment using multiple tubes to connect the pump to the fluid bag.

FIG. 10 is a view similar to FIG. 9 with the tubes connected to the fluid bag spaced apart.

FIG. 11 is a schematic drawing showing the pump connected to the fluid bag with a V-fitting.

FIG. 12 is a view showing the pump of FIG. 11 removed from the support bracket.

FIG. 13 is a schematic drawing showing the pump connected to the fluid bag with a Y-fitting.

FIG. 14 is a schematic drawing showing the Y-fitting of FIG. 13.

FIG. 15 is a cross-sectional view of the Y-fitting shown in FIG. 14.

FIG. 16 is a schematic drawing showing the pump connected to the fluid bag with a W-fitting.

FIG. 17 is a cross-sectional view of the W-fitting of FIG. 16.

FIG. 18 is a cross-sectional view similar to FIG. 17 with flexible tubes connected to the W-fitting.

FIG. 19 is a schematic view of an alternative support for the pump including a micro switch.

FIG. 19A is a partial schematic view of the micro switch of FIG. 19 in an open position.

FIG. 19B is a view similar to FIG. 19A with the micro switch in a closed position.

FIG. 20 is a schematic view of another embodiment with a hanging support including a gravity switch.

FIG. 21 is a partial cross-sectional view of the gravity switch.

FIG. 22 is a schematic view showing the pump supported by a hanging sling.

FIG. 23 is a partial cross-sectional view of a modified impeller housing including a slide valve shown in an open position.

FIG. 24 is a view similar to FIG. 23 showing the slide valve in a closed position.

FIG. 25 is a schematic drawing of the slide valve used in FIGS. 23 and 24.

FIG. 26 is a schematic drawing of a fluid bag supported in a carriage.

FIG. 27 is a schematic drawing of the pump supported by an L-bracket.

FIG. 28 is a schematic drawing similar to FIG. 27 including a spacer to support a fluid bag.

FIG. 29 is a partial schematic drawing showing a fluid bag supported by the spacer of FIG. 28.

FIG. 30 is a partial exploded schematic drawing showing the connection of the impeller housing to the L-bracket.

FIG. 31 is a schematic assembly drawing of the device shown in FIG. 30.

FIG. 32 is a partial cross-sectional assembly view of FIG. 31.

FIG. 33 is a partial cross-sectional assembly view showing an alternative assembly of the impeller housing to the L-bracket.

FIG. 34 is a partial cross-sectional view of the pump mounted to the L-bracket by a bayonet lock.

FIG. 35 is a partial cross-sectional view of an alternative connection of the pump to the fluid bag by a friction fit connector.

FIG. 36 is a partial schematic drawing showing an another embodiment including a plunger valve.

FIG. 37 is a partial cross-sectional view of an embodiment in the pump includes a detent mechanism to start or stop fluid flow with the mechanism in a closed position.

FIG. 38 is a view similar to FIG. 37 with the mechanism in an open position.

FIG. 39 is a partial schematic drawing showing the pump supported from the bag by a retainer fan.

FIG. 40 is a partial schematic drawing showing the actuator for the retainer fan with the retainer fan in an open position.

FIG. 41 is a partial schematic drawing similar to FIG. 40 with the retainer fan in a retracted position.

### DETAILED DESCRIPTION OF THE INVENTION

The present invention is useful in surgical irrigation systems and encompasses a large number of alternative designs. The system includes a novel assembly for a pumping mechanism, a pump support, and a fluid source, to provide fluid flow to a surgical instrument, such as a lightweight handpiece. The invention provides a fluid pump assembly that is simple, efficient, and safe.

One aspect of the surgical irrigation system of this invention provides a safer and more sterile irrigation system by providing a pump and fluid source assembly in which fluid connection between the pump and fluid source is only possible if the pump is clamped to the IV pole or otherwise supported. By limiting the establishment of fluid flow between the pump and the fluid source to only those times when the pump is supported the present invention reduces the chance that the pump will become disconnected from the fluid source during a surgical procedure.

Another aspect of this invention provides alternative connections between the pump and the fluid source in which fluid flow to the pump is blocked until a user performs a function that activates fluid flow. Similarly, such connection may discontinue fluid flow if the pump system is not properly set up.

To fully understand the breadth of the present invention, reference is made to non-limiting examples of particularly preferred embodiments of the present invention. They are described below with reference to the figures.

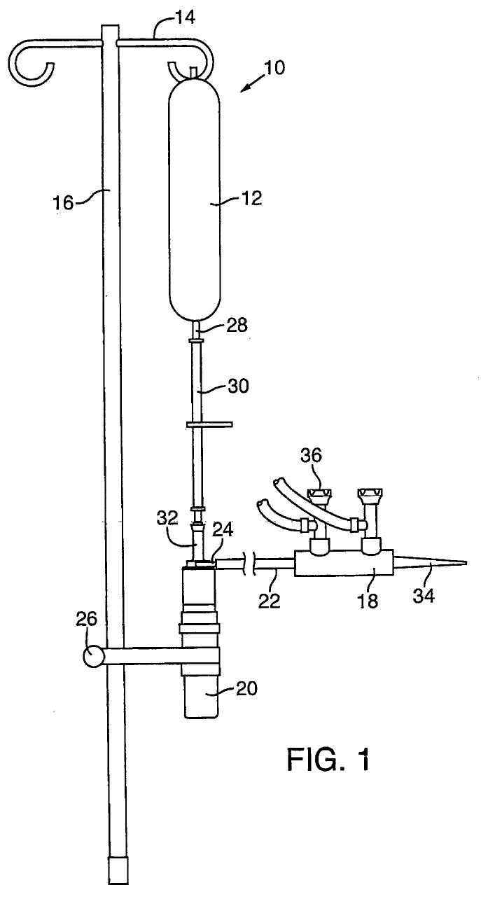

A representative surgical irrigation system of the present invention is illustrated in FIG. 1 which shows a fluid source 10, e.g., bag 12, that may be located in a conventional manner, e.g., by utilizing a bag 12 containing fluid supported by a horizontal arm 14 adjustably fixed on a vertically standing pole 16 in a manner such that bag 12 is spaced apart from and at a level above a surgical handpiece 18. The fluid is supplied to handpiece 18 through a pump 20 by providing a flexible conduit such as elongate flexible tubing 22 running from a pump outlet 24 to handpiece 18. Pump 20 is connected to bag 12 via a flexible tube 30 and need not be located on handpiece 18. Pump 20 is preferably adjustably fixed and supported on vertically standing pole 16 by a support bracket 26. Bag 12 includes an outlet 28 connected by a flexible tube 30 to an inlet 32 on pump 20.

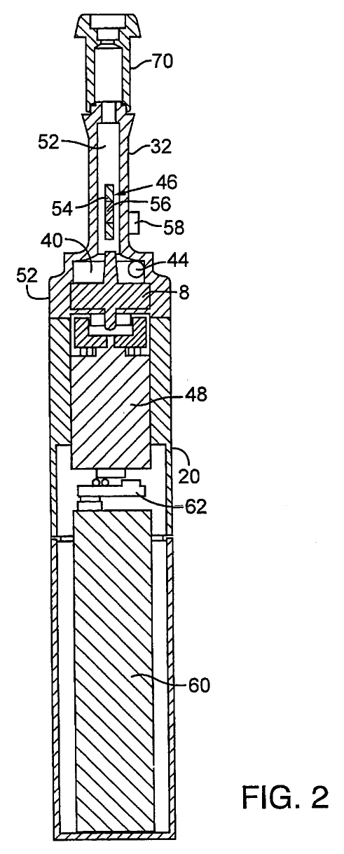

Handpiece 18 is one type of surgical apparatus that may be used with the irrigation system of this invention. Handpiece 18 is supplied with probe 34, capable of entering into a surgical site, allowing the surgeon to deliver fluid to the surgical site. Handpiece 18 may be of the type shown and described in U.S. Pat. No. 6,176,847, the disclosure of which is herein incorporated by reference. Handpiece 18 may also be supplied with a hand controllable valve 36, here shown as a trumpet valve, capable of starting and stopping gravity flow of fluid from bag 12, through pump 20. The surgeon opens valve 36 to start gravity flow of fluid. Pump 20 may then be activated so that an impeller or fluid accelerator 38 (FIG. 2) increases fluid flow to handpiece 18.

The surgeon may stop fluid flow by simply closing valve 36 on handpiece 18. The remotely located pump 20 may then act to turn off and stop fluid flow as will be described in further detail.

Handpiece 18 may also be connected to a suction source, and handpiece 18 can be designed to allow the surgeon to easily select between supplying fluid, such as irrigation liquid, or vacuum to a surgical site, using connecting tubing shown leading into the two trumpet valves shown in FIG. 1.

As seen in FIG. 2, pump 20 can be generally characterized as having a fluid flow chamber 40 to which fluid flows through inlet 32 connected to a fluid bag or other source of fluid. Within the fluid flow chamber 40 is located a suitable flow detector 46 which is sensitive to fluid flow. Flow detector 46 may additionally comprise a conventional or proprietary trumpet valve for opening or closing gravity flow. A motor 48 is provided with an activator to be described in detail, to activate motor 48 that in turn drives a pump or impeller to increase fluid flow.

Impeller housing 50 includes inlet 32 having an inlet passage 52 containing a buoyant capsule 54 which additionally carries a magnet 56. Magnet 56 is shown located within capsule 54 but may be located on an outer surface thereof, or elsewhere. Buoyant capsule 54 is located within fluid flow chamber 40. A Hall-Effect switching circuit board and Hall-Effect sensor 58 are attached to pump 20 but outside of fluid flow chamber 40 and at a selected distance below inlet passage 52. Hall-Effect switching circuit is connected to motor 48 by conventional electric wires (not shown). Motor 48 is powered by any conventional means, for example, by batteries 60 operating through circuit board 62. Outlet port 44 of outlet 24 is connected by fluid flow tubing to surgical handpiece 18, which carries valve 36 for starting and stopping fluid flow. Since pump 20 of FIG. 2 utilizes a buoyant capsule 54 as a means that swings up and down in response to fluid flow, it is preferable that during operation, the device be arranged in an at least partial vertical position.

As one skilled in the art will now understand, as a trickle of fluid enters inlet passage 52, buoyant capsule 54 is displaced downward toward the bottom of fluid flow chamber 40. As buoyant capsule 54 reaches a position in fluid flow chamber 40 substantially even with the location of Hall-Effect sensor 58, magnet 56 located on or within buoyant capsule 54 activates the Hall-Effect circuit, which in turn activates motor 48. Motor 48 then drives impeller 38. As impeller 38 turns the flow within fluid flow chamber 40 is accelerated to the desired flow rate. When the surgeon wishes to stop the flow of fluid, the surgeon needs no electric switch but merely has to close valve 36 on handpiece 18. Flow within fluid flow chamber 40 is then stopped and buoyant capsule 54 rises again to the upper portion of fluid flow chamber 40, thus deactivating the Hall-Effect switching circuit and motor 48.

Preferably, buoyant capsule 54 is large enough in diameter so that when fluid flow is stopped, and buoyant capsule 54 rises to the top of fluid flow chamber 40, buoyant capsule 54 is capable of providing a seal around inlet passage 52 to prevent backflow of fluid out of fluid flow chamber 40. Additionally, it is sometimes desirable to include ribs, protuberances or the like spaced about the inner surface of fluid flow chamber 40 at the lower end thereof to prevent buoyant capsule 54 from sealing off fluid flow through the bottom portion of fluid flow chamber 40.

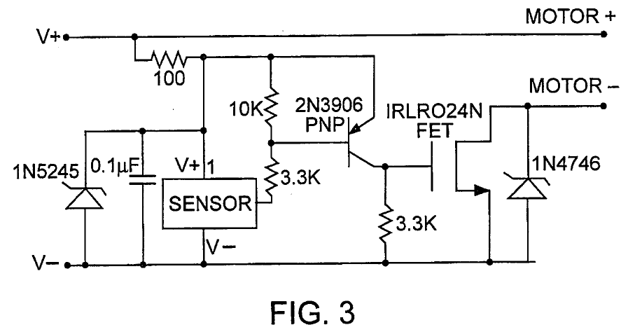

FIG. 3 is a circuit diagram showing one of many appropriate components and one preferred way they may be connected to a motor for battery operation between voltages V+ and V−. The word “sensor” in the diagram may preferably be a “Hall-Effect” sensor as previously described.

In one preferred embodiment as seen in FIG. 1, the pump 20 is supported on the pole 16 by a bracket 26 that is adjustably clamped to the pole 16 in a conventional manner. The bracket 26 may include a through opening having a diameter slightly smaller than the diameter of the pump housing so that the pump is snugly received in the through opening and held by the bracket 26. Alternatively, the bracket 26 may support the pump 20 housing by a cup-shaped support as seen in FIG. 5.

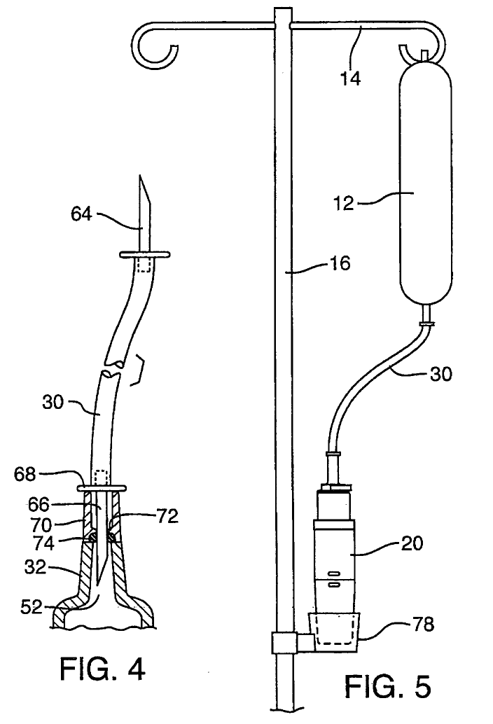

In the embodiment of FIG. 1, single extension tube 30 connects pump 20 to fluid bag 12. As seen more clearly in FIG. 4, tube 30 includes spike connectors 64 and 66 sealingly attached to opposite ends of tube 30. Spike 64 is adapted to be connected to fluid bag 12 through a typical luer connector. Spike 66 is adapted to be connected to inlet 32 so that the end of spike 66 is located within the upper portion of inlet passage 52. Each spike 64 and 66 includes an annular rim 68 as seen on spike 66 that serves as an abutment for the end of tube 30. Additionally, rim 68 abuts the top of the inlet 32 to provide a tight and secure connection.

Inlet 32 may include a receptacle 70 ultrasonically welded or otherwise permanently attached to the top of inlet 32 for connection with spike 66. The receptacle is hollow to accommodate spike 66 and includes an inner shoulder 72 that snugly receives spike 66. An O-ring seal 74 is located within inlet passage 52 just below annular shoulder 72 to provide a fluid tight seal.

As seen in the embodiment of FIG. 1, bracket 26 is adjusted so that pump 20 may be aligned with or located directly under fluid bag 12. Tube 30 is then connected to pump 20 through spike 66 and to fluid bag 12 by spike 64. A clip 76 is preferably located centrally of tube 30 to block fluid flow and prevent automatic flow of fluid from fluid bag 12 to pump 20 once tube 30 has been connected to them. When it is desired to start fluid flow clip 76 is removed allowing free and unimpeded flow of fluid from fluid bag 12 to pump 20.

Inlet 32, including receptacle 70, is designed to mate only with a standard spike 66 and is not designed to mate with a standard fluid bag outlet. The connection of spike 66 to inlet 32 is constructed and arranged to provide a fluid tight engagement; however, the coefficient of friction between the inner surfaces of inlet 32 such as, for example, inner shoulder 72 and spike 66 are lower than that required to support the weight of pump 20. Therefore, if pump 20 is not supported by bracket 26 the weight of pump 20 will cause it to disengage from spike 66 and pump 20 will fall. The requirement that pump 20 be supported in order to maintain a fluid connection substantially reduces the likelihood that the pump will inadvertently become disconnected from the fluid bag resulting in fluid loss and creating unsanitary conditions and possibly danger to the patient. Spike 66 is shown in FIGS. 1 and 4 as being connected to receptacle 70; however, spike 66 may be connected directly to inlet 32.

Pump 20 is shown in FIG. 1 as being supported directly under fluid bag 12. However, as seen in FIG. 5 tube 30 allows pump 20 to be located closer to pole 16. Arm 14 may be extended from the position shown in FIG. 1 so that fluid bag 12 is vertically spaced from pump 20. Alternatively, pump 20 may be supported at a closer location to pole 16 using a modified bracket 78. Supporting pump 20 at a spaced distance from fluid bag 12 provides more open room for the surgeon and further reduces the chance that the surgeon or other personnel will bump or otherwise cause pump 20 to become disconnected from fluid bag 12.

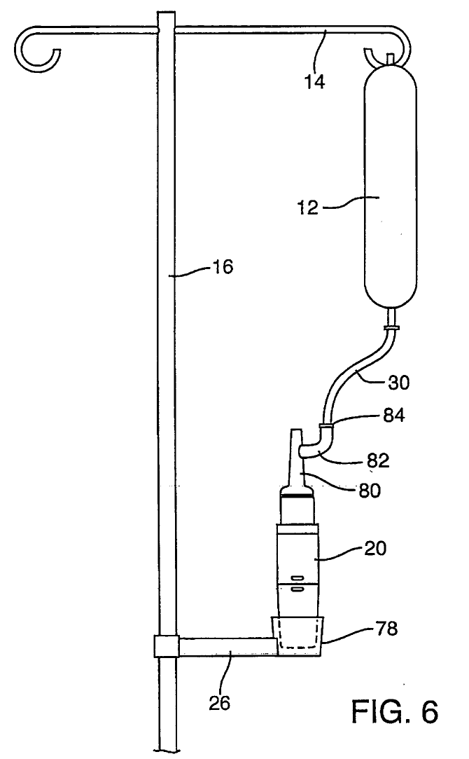

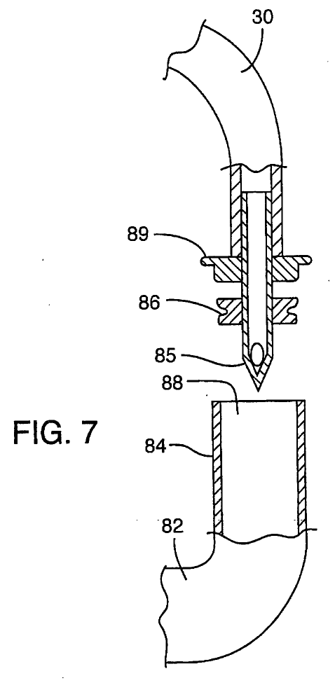

FIGS. 6 and 7 show another embodiment to further accommodate location of pump 20 at a space location from the fluid bag 12. In this embodiment, impeller housing 80 is modified to include an inlet connector 82 that extends laterally from impeller housing 80 and includes a generally upturned end 84 for connection with spike connector 85. Spike 84 preferably includes a seal 86 adapted to be received in passageway 88 and a seal 89 that sealingly engages the top of passageway 88 as seen in FIG. 7.

FIG. 8 shows another embodiment that allows pump 20 to be located at a spaced location from fluid bag 12. In this embodiment, pump 20 is supported on arm 14 on an opposite side of pole 16 than fluid bag 12. Impeller housing 90 includes a support loop 92 that is adapted to be hung from arm 14a. Impeller housing 90 further includes an inlet connector 94 that extends laterally from impeller housing 90 for connection with tube 30. This connection may be through a press friction fit as shown or may be through a conventional spike connector.

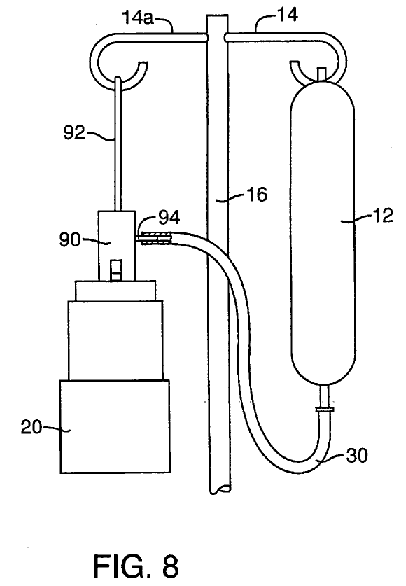

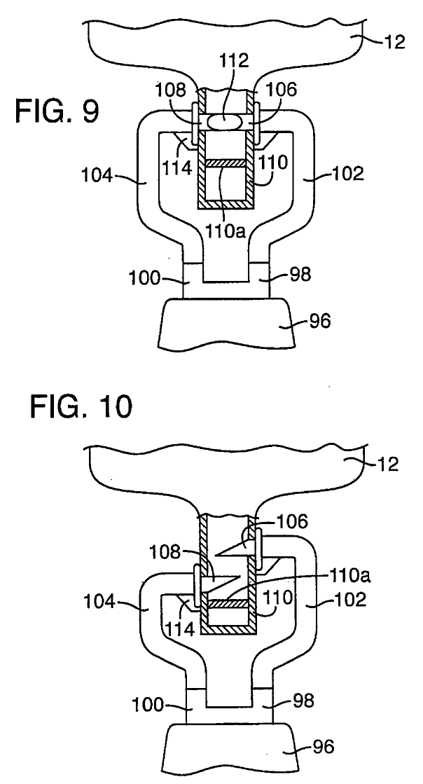

FIGS. 9 and 10 show alternative embodiments for connecting pump 20 to fluid bag 12. Impeller housing 96 includes dual inlet connectors 98 and 100 for connection with separate flexible tubes 102 and 104. Tubes 102 and 104 each include spikes 106 and 108, respectively, that are adapted for mating engagement with each other. Spikes 106 and 108 are pressed into opposed sides of fluid bag outlet 110 above seal 110a so that the ends of spikes 106 and 108 are in mating engagement. Spikes 106 and 108 are constructed and arranged so that when they are in mating engagement their ends form an opening 112 to allow fluid to flow from fluid bag 12 to tubes 102 and 104 and into pump 20. Each spike 106 and 108 includes an outside seal 114 to prevent fluid leakage outside of fluid bag 12. Using this system, the spike need not be compatible with a standard size bag outlet.

FIG. 10 shows an embodiment similar to the one shown in FIG. 9 except that spikes 106 and 108 are pressed into opposed sides of fluid bag outlet 110 above seal 110a at vertically spaced locations so that the ends of spikes 106 and 108 are not matingly engaged but are located in a spaced apart manner so that fluid flows into each spike separately.

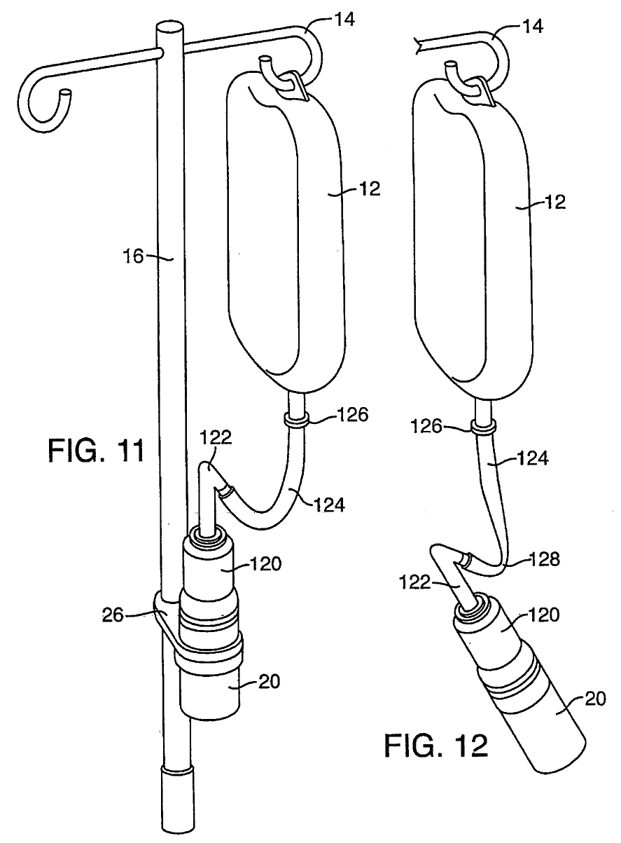

FIGS. 11 and 12 show another embodiment for connecting a supported pump to a fluid source. In this embodiment, impeller housing 120 includes a hollow V-fitting 122 secured thereto at one end. Tube 124 is secured to the other end of V-fitting 122 and includes a standard spike at its opposite end for connection with fluid bag 12. The V-fitting 122 allows pump 20 to be located at a position other than directly under fluid bag 12 and as long as pump 20 is supported by bracket 26 fluid freely flows from fluid bag 12 to pump 20. However, as seen most clearly in FIG. 12, if pump 20 is removed from bracket 26 tube 124 becomes pinched at a central portion 128 and obstructs fluid flow. This advantageously prevents fluid flow if the pump is not properly supported in its bracket

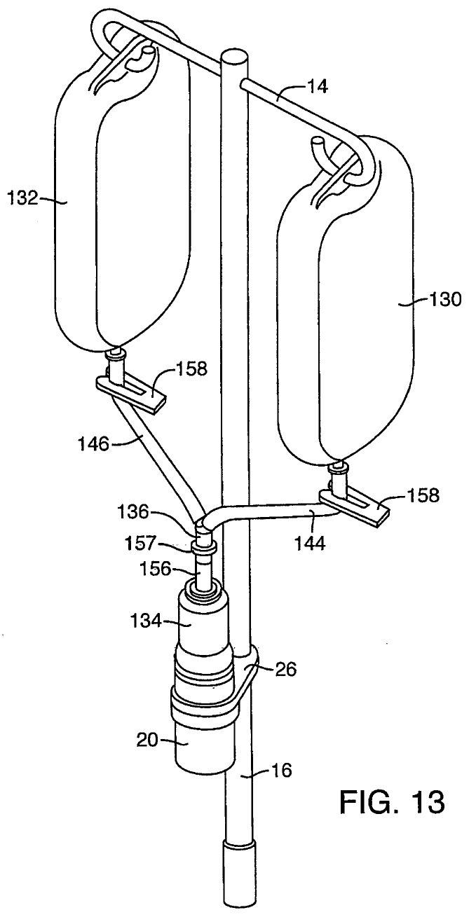

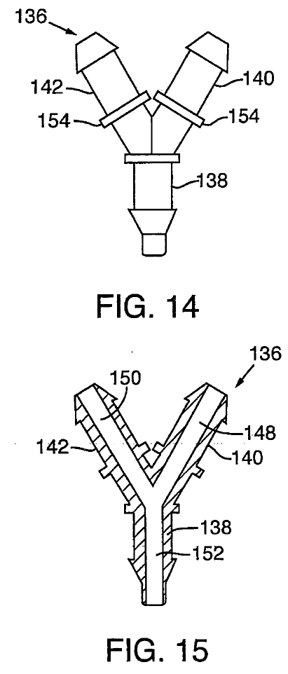

FIGS. 13-15 show a supported pump 20 capable of connection with two fluid bags 130 and 132 to allow for a surgical procedure to continue uninterrupted during changing of the fluid bags. In this embodiment, impeller housing 134 includes a Y-fitting 136 connected thereto. Y-fitting 136 includes a base connector 138 adapted for insertion into the inlet of impeller housing 134. Two divergent connectors 140 and 142 are each adapted for connection to a fluid bag 103 and 132 with a tube 144 and 146, respectively. As seen in FIGS. 14 and 15, Y-fitting 136 is hollow forming fluid passageways so that passageways 148 and 150 open into passageway 152 of the base connector 138. Base connector 138 has a shape that substantially corresponds to the inlet connector of impeller housing 134 to provide a sealing engagement therewith. Divergent connectors 140 and 142 are connected to their respective tube 144 and 146 by a press or friction fit so that the end of each tube abuts annular rim 154. Impeller housing 134 includes a blunt-type fitting 156 for connection with Y-fitting 136. A receptacle 157, similar to receptacle 70 described above, may be ultrasonically welded or mechanically bonded to blunt fitting 156. This joint includes a lubricious seal or O-ring similar to that shown and described in FIG. 4 which will be placed in after welding or bonding.

As shown in FIG. 13, pump 20 is supported in bracket 26. Tubes 144 and 146 are then connected to their respective fluid bag 130 and 132 through a conventional spike. Each tube 144 and 146 preferably includes a clamp 158 that pinches the tubes or otherwise blocks fluid flow once the tubes are connected to their respective fluid bag. When fluid flow is desired one of clamps 158 is removed and fluid freely flows from one of the fluid bags to pump 20 through Y-fitting 136. When the one fluid bag empties clamp 158 on the other tube is removed and fluid then freely flows from the other bag while the one fluid bag is replaced, if necessary.

The contact surfaces between Y-fitting 136 and tubes 144 and 146 may be designed so that if pump 20 is removed from bracket 26 pump 20 will separate from the Y-fitting 136 under its own weight or some other predetermined force. This allows for easy changing of pump units. This is because Y-fitting 136 and the associated O-ring seal are designed to have surface or joint contact but the O-ring lubricity does not allow for frictional forces higher than the weight of the pump.

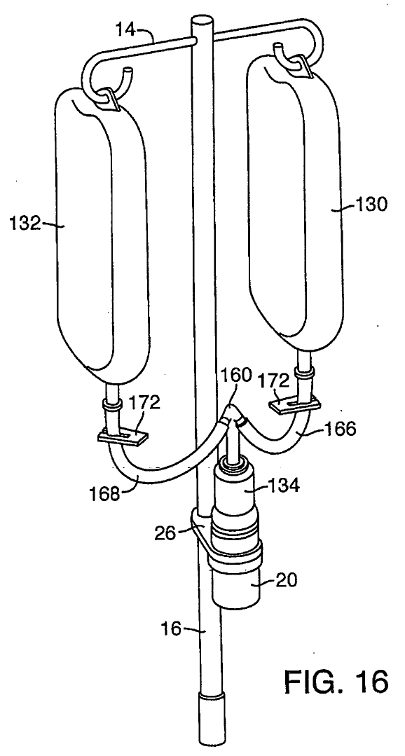

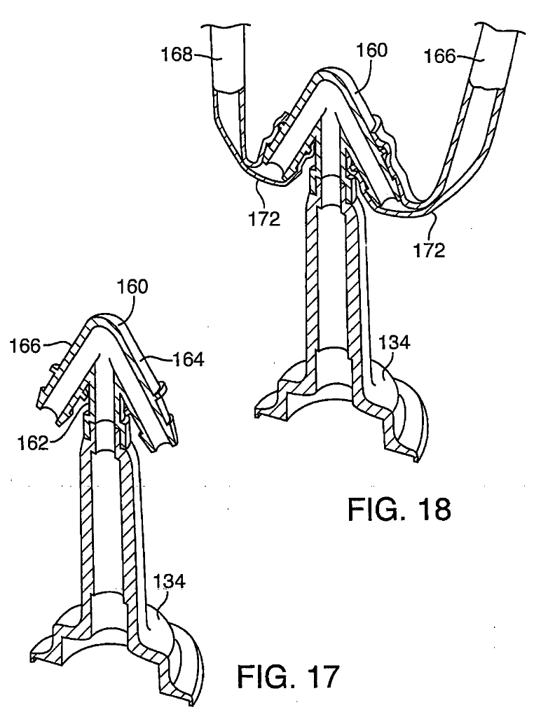

FIGS. 16-18 show another embodiment in which pump 20 is adapted for connection to two fluid bags 130 and 132. In this embodiment, impeller housing 134 includes a W-fitting 160 bonded or otherwise permanently secured to impeller housing 134. W-fitting 160 includes a base connector 162 having divergent downwardly extending connectors 164 and 166 each adapted for connection with a flexible tube 168 and 170, respectively. Each tube 168 and 170 includes a standard distal end spike permanently bonded thereto for connection to a fluid bag. W-fitting 160 is hollow to allow for free flow of fluid from tubes 168 and 170. The operation of this embodiment is similar to that described with reference to the embodiment of FIG. 13 wherein pump 20 with W-fitting 160 is supported by bracket 26. Tubes 168 and 170 are then connected to their respective bags and when fluid flow is desired one of the clamps 172 is removed from its associated tube. As previously described with reference to FIG. 13, when one fluid bag is empty clamp 172 on the other tube is removed to allow fluid flow from the other fluid bag. The one fluid bag can then be replaced, if necessary.

This embodiment allows pump 20 to be connected to two fluid bags at once but requires that the pump be located within or held by bracket 26 in order to provide fluid flow and operate properly. When pump 20 is held by bracket 26 and adjusted to the desired position tubes 168 and 170 provide for the free flow of fluid from their associated fluid bag x to pump 20 as long as clamp 172 is removed. However, should pump 20 be removed from bracket 26 and allowed to hang by its own weight tubes 168 and 170 will pinch as seen at 172 in FIG. 18 to block fluid flow.

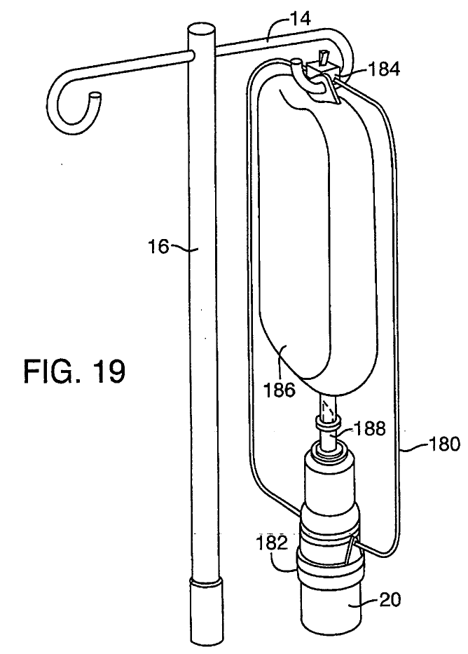

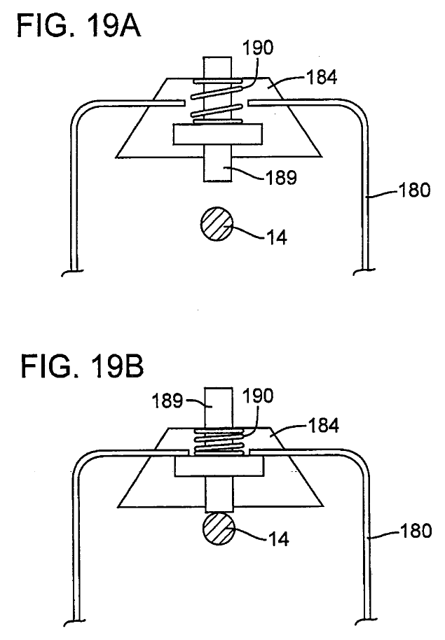

FIG. 19 shows another embodiment in which pump 20 must be supported in order to operate properly. In this embodiment, pump 20 is supported in a carriage 180 that includes a holder 182 to support pump 20. Carriage 180 further includes a micro switch 184 constructed and arranged so that when pump 20 is located in holder 182 and connected to fluid bag 186 by a standard spike connection 188 micro switch 184 completes an electrical circuit providing power to 20 through carriage 180 to the pump motor.

As seen in FIGS. 19A and 19B, the micro switch 184 includes a spring-biased plunger 189 that comprises an electrical contact and is normally biased by spring 190 to an open position (FIG. 19A). When carriage 180 is suspended from arm 14 plunger 189 is moved against the bias of spring 190 to close (FIG. 19B) and make contact with carriage 180 to complete an electrical circuit to provide power to the pump motor.

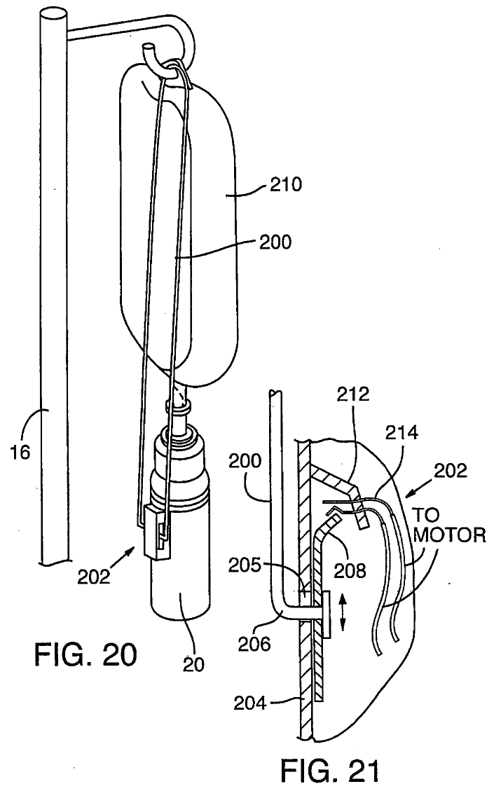

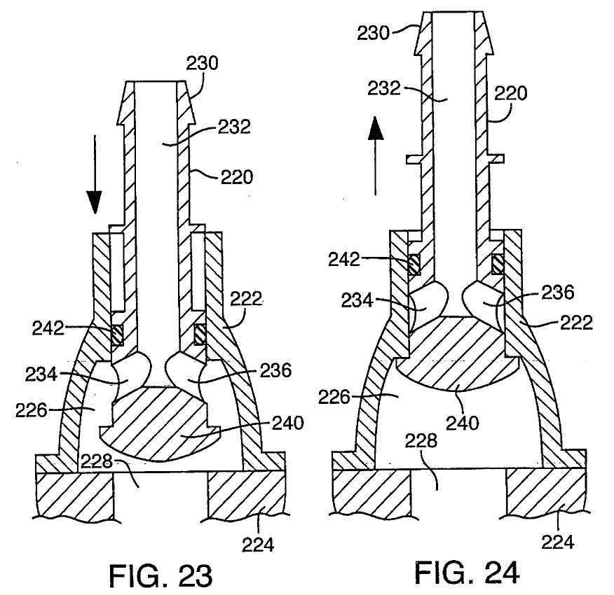

FIGS. 20 and 21 show another embodiment in which pump 20 is supported by a hanging rod 200 that includes a gravity switch 202 located on pump housing 204. As seen most clearly in FIG. 23, hanging rod 200 includes inwardly turned end portions 206 that extend through pump housing 204 through an elongated slot 205 that slidably receives end 206. A slide 208 is securely fixed to one end 206 just inside of pump housing 204 and comprises an electrical contact. Pump 20 is attached to hanging rod 200 so that after pump 20 is connected to fluid bag 210 pump 20 descends under its own weight so that pump housing 204 slides relative to the electrical contact 208 until it abuts inward extension 212 and makes contact with wires 214 leading to the pump motor. This completes the electrical circuit and provides power to pump 20.

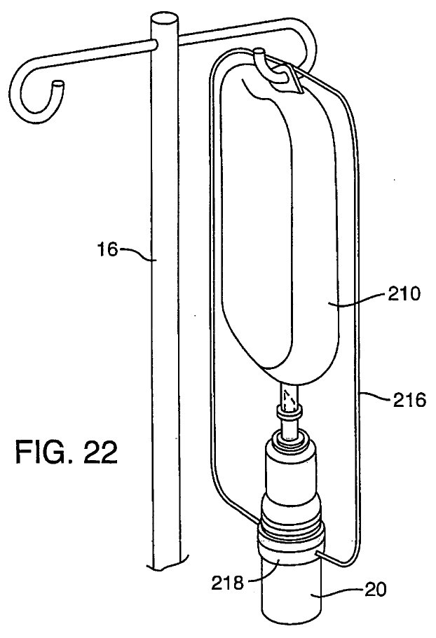

FIG. 22 shows another embodiment in which pump 20 is supported in a hanging sling 216 suspended from pole 16. Pump 20 is supported by a central section 218 that includes an opening to snugly receive pump 20.

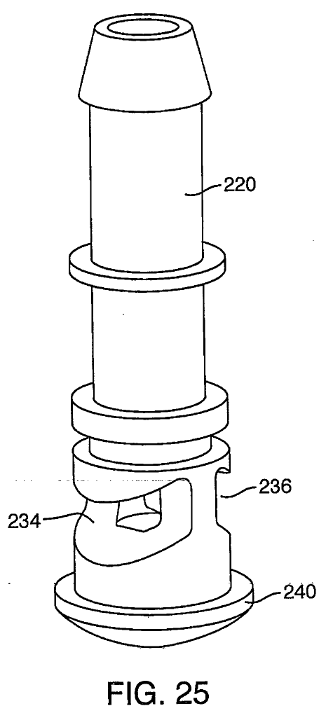

FIGS. 23-25 show an embodiment in which the pump is directly connected to a fluid bag by a slide valve 220 that is open as long as the pump is supported in a bracket but which closes if the pump is removed from the bracket and allowed to be suspended from the fluid bag. In this embodiment, slide valve 220 is located in retainer housing 222 that is permanently secured to impeller housing 224. Retainer housing 222 includes a fluid passageway 226 that is open to passageway 228 in impeller housing 224. When slide valve 220 is in an open position fluid flows through slide valve 220 into fluid passageways 226 and 228 and on to the pump. Slide valve 220 includes an O-ring seal 242 for sealing engagement with retainer housing 222.

Slide valve 220 includes a stepped tubular portion 230 for connection to a fluid bag. Connecting slide valve 220 to the fluid bag causes slide valve 220 to be moved downward relative to retainer housing 222 to the position shown in FIG. 23. As long as the pump is supported under the fluid bag slide valve 220 will remain in the open position shown in FIG. 23. In this position fluid flows from the fluid bag into a passageway 232 and through ports 234 and 236 into fluid passageways 226 and 228. However, if the pump is removed from the bracket and allowed to hang from the fluid bag the pump will fall due to its own weight and retainer housing 222 will slide relative to slide valve 220 until stop member 228 on the lower end of slide valve 220 engages the upper wall 240 of retainer housing 222 to close ports 234 and 236 as seen in the closed position of FIG. 24.

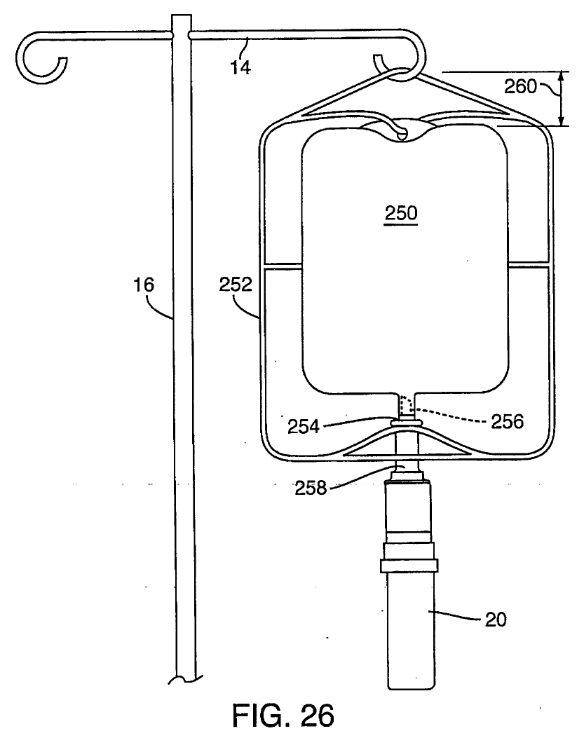

FIG. 26 shows a system in which pump 20 is connected to a fluid bag 250 that is suspended inside a carriage 252 hung from a standard arm 14. A standard spike 254 is inserted into fluid bag 250 having a standard outlet. Spike 254 is then connected to an inlet fitting 258 on pump 20. Fluid bag 250 is positioned within carriage 252 so that a gap 260 exists between arm 14 and fluid bag 250 so that it will pull away from pump 20 if fluid bag 250 is hung directly and independently from arm 14.

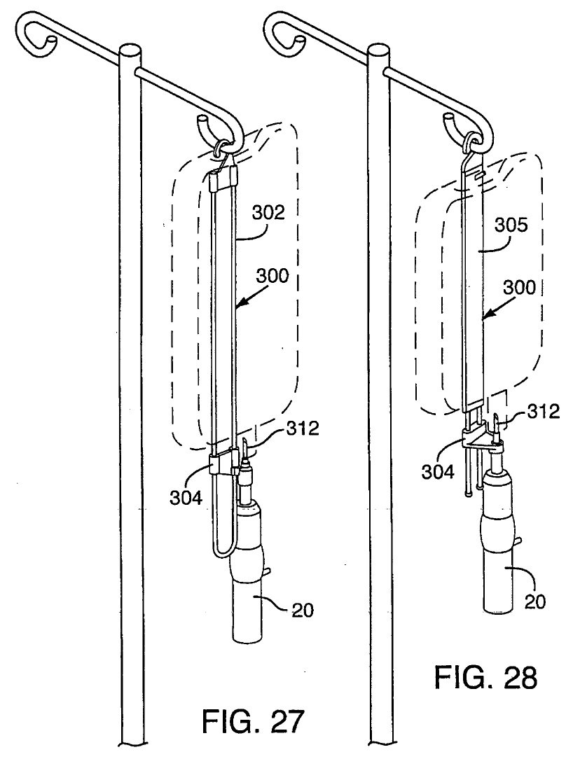

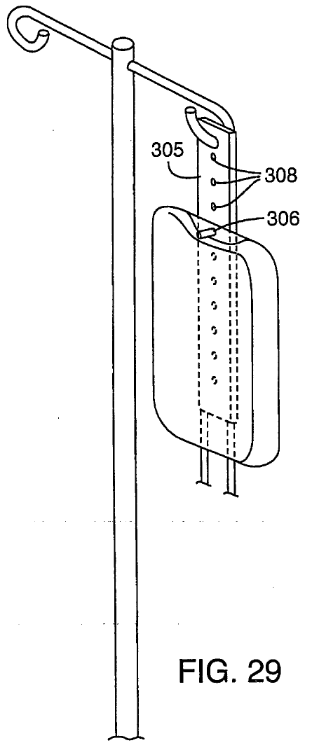

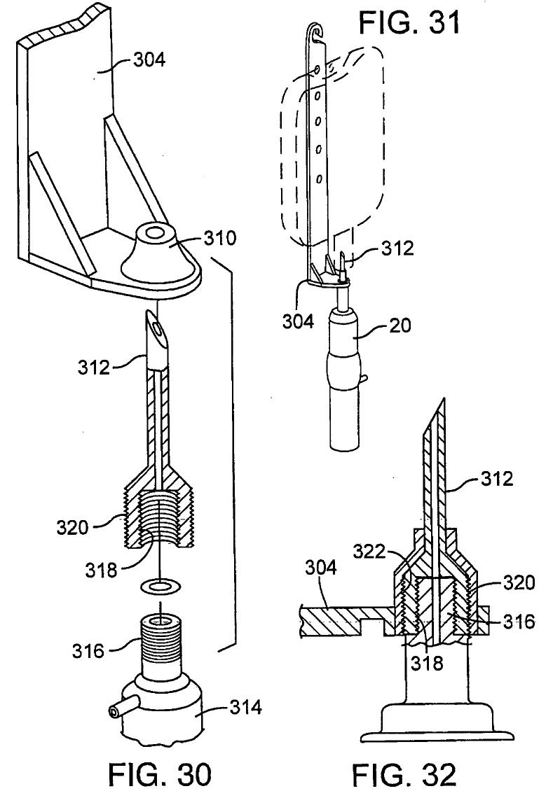

FIGS. 27-32 show an alternative system of supporting a pump connected to a fluid bag. In this embodiment, an L-bracket 300 is provided to hook onto a standard pole. The L-bracket 300 includes an elongate body 302 that adjustably supports an extension member 304 that supports pump 20. In FIG. 27 extension member 304 is adjustable along the length of body 302 to connect to various size fluid bags. FIGS. 28 and 29 show one embodiment of a support member 305 to which a fluid bag may be supported by inserting a pin 306 (FIG. 29) in one of a number of holes 308 in support member 305. Alternatively, a fluid bag may hung from hooks or prongs along body 302. Pump 20 is connected to the fluid bag by a spike 312. Extension member 304 includes a connector portion 310 to which both pump 20 and spike 312 are attached by a threaded connection. Impeller housing 314 includes outer threads 316 for threaded engagement with inner threads 318 on spike 312. Spike 312 further includes outer screw threads 320 for threaded engagement with inner screw threads 322 on connector portion 310.

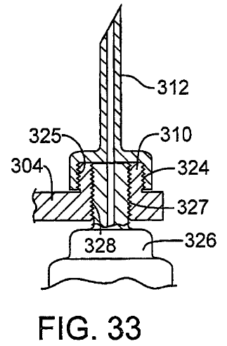

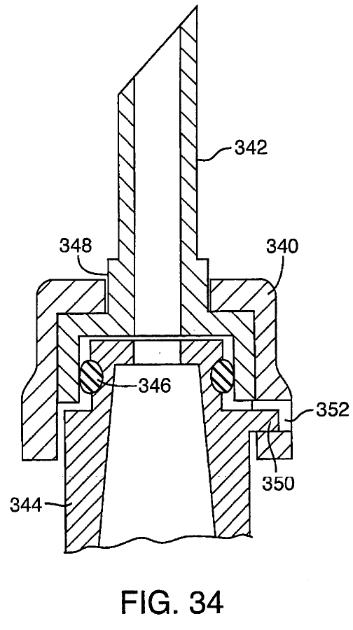

In an alternative embodiment of FIG. 33 connector portion 310 may include outer screw threads 324 for threaded engagement with inner screw threads 325 on spike 312 so that spike 312 is screw threaded onto the top of connector portion 310. Impeller housing 326 includes outer threads 327 for threaded engagement with inner screw threads 328 located in connector portion 310.

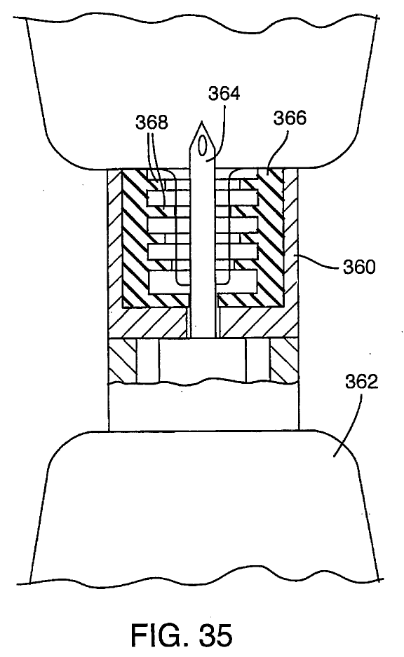

Instead of screw threads the pump may be connected to the L-bracket extension 340 by a bayonet-type lock as seen in FIG. 35. In this embodiment, spike 342 is connected to a top end of the impeller housing 344 by a press fit with an O-ring 346 providing a fluid seal. Impeller housing 344 is located within extension 340 so that spike 342 extends through an opening 348 for connection with a fluid bag. Impeller housing 344 is then rotated so that a protrusion 350 is received in a lock opening 352 in extension 340.

In this embodiment the L-bracket and spike 342 are re-usable but must first be sterilized before each use. However, the pump and spike 342 may also be a disposable as a single use entity. In either case, the pump is not capable of attaching to the fluid bag without the use of the L-bracket.

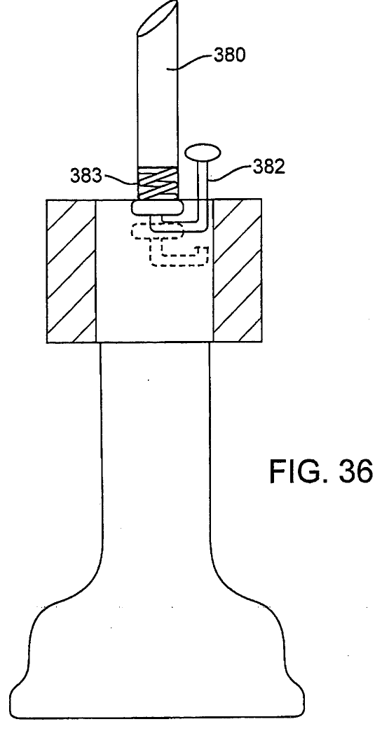

FIG. 36 shows another embodiment in which a pump such as, for example, is directly connect to a fluid bag and is supported by a special fitting. An outer fitting 360 is securely attached to impeller housing 362 so that spike 364 extends through and out of outer fitting 360 for connection with a fluid bag. Outer fitting 360 includes an inner member 366 with gripping teeth 368 to frictionally grip the outside of fluid bag outlet 365 when spike 364 is attached thereto. In this embodiment, the pump is suspended from the fluid bag but is held by the frictional engagement between inner member 366 and the fluid bag outlet.

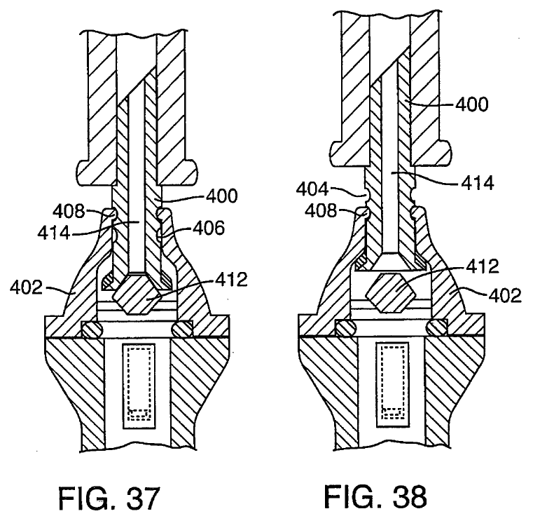

FIG. 37 shows another embodiment in which the pump is suspended from the fluid bag by a spike 380. However, fluid flow is not automatically started upon spiking the fluid bag. Spike 380 includes a valve lever 382 that is normally biased to a closed position by an extension spring 383. After spike 380 has been inserted into the fluid bag the fluid bag presses down on valve lever 382 against the bias of extension spring 383 to the position shown in phantom in FIG. 37 to open the fluid passageway in spike 380 to allow fluid to flow from the fluid bag. Fluid flow will only be facilitated when valve lever 382 is moved down by the fluid bag. When the fluid bag is removed valve lever 382 is closed by extension spring 383.

Alternatively, spike 380 may be non-tubular and may sealingly engage the fluid bag so that the pump hangs independently from the fluid bag by spike 380 and valve 382 may be part of impeller housing.

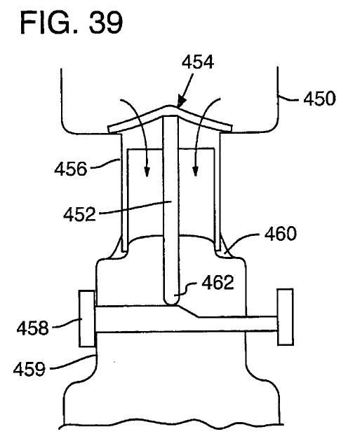

FIGS. 38 and 39 show another embodiment in which the pump is suspended from the fluid bag by a spike 400. Retainer housing 402 slidably supports spike 400 that includes upper and lower detent recesses 404 and 406 for selective engagement with detent 408 on retainer housing 402. A stopper 412 is fixedly located within retainer housing 402. After spike 400 is connected to the fluid bag spike 400 is moved into the sealed position shown in FIG. 38 in which detent 408 is located in upper recess 404. In this position, stopper 412 closes fluid passageway 414 to block fluid flow. The user is then required to pull the pump downwardly to move detent 408 into lower recesses 406 to open passageway 414.

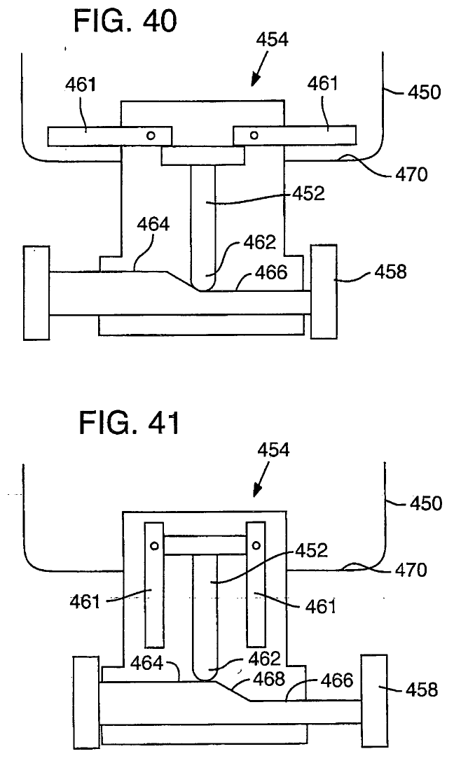

FIG. 40 shows an alternative embodiment of supporting the pump from a fluid bag 450. In this embodiment, spike 452 includes a retainer fan 454 that is retracted until spike 452 is inside fluid bag 450. Spike 452 has an outer diameter that is less than the inner diameter of fluid bag outlet 456 so that there is clearance between the walls of fluid bag outlet 456 and spike 452. In use, the user will spike fluid bag 450 while retainer fan 454 is retracted. After spiking fluid bag 450, retainer fan 454 is opened by an actuator 458 on impeller housing 460 so that retainer fan 454 moves up into fluid bag 450 and opens. Retainer fan 454 will be completely open when it passes fluid bag outlet 456. The user then releases the pump which will drop under its own weight so that retaining fan 454 can rest on the inside of fluid bag 450. The fluid bag outlet 456 includes a seal 462 to prevent fluid loss.

FIGS. 41 and 42 show one embodiment for actuating retainer fan 454. Retainer fan 454 includes spring-biased arms 460 that are normally closed as seen in FIG. 42. To open retainer fan 454 actuator 458 is moved to the left as shown in FIG. 42 so that bottom portion 462, which in the closed position rests on upper surface 464 of actuator 458, moves downward along surface 468 to rest on surface 466. As bottom portion moves arms 460 are biased open (FIG. 41) to rest or engage inner surface 470 of fluid bag 450.

What is claimed:

1. A surgical irrigation system, comprising:
a fluid source having an outlet to supply fluid to a pump device, the pump device being constructed and arranged so that fluid connection between the fluid source and the pump device is established only when the pump is held by a support.

2. The system of claim 1 in which;
the pump device is connected to the fluid source by a flexible tube through a friction fit in which the coefficient of friction is less than that required to support the weight of the pump.

3. The system of claim 1 in which,
the pump device includes an impeller housing with an inlet connection adapted for connection with a tube the inlet cooperating with the tube to provide a fluid flow path from the fluid source to the pump.

4. The system of claim 3 in which the inlet connection extends laterally from the impeller housing.

5. The system of claim 4 wherein the impeller housing further includes a hook for suspension from a pole.

6. The system of claim 1 in which the impeller housing includes dual inlets for connection with separate tubes, wherein the tubes are connected to opposite sides of the fluid source outlet

7. The system of claim 6 wherein the tubes include spikes connected to the fluid source above a seal.

8. The system of claim 6 wherein the tubes include spikes having ends that are matingly engaged when inserted into the fluid source.

9. The system of claim 6 wherein the tubes are connected to the fluid source outlet at vertically spaced locations.

10. The system of claim 1 further including a pinching device for providing a fluid connection between the fluid source and the pump.

11. The system of claim 10 wherein the pinching device is a V-fitting connected to the impeller housing having a tube bonded thereto being constructed and arranged so that the tube is pinched to block fluid flow if the pump device is removed from the support.

12. The system of claim 9 wherein the pinching device is a Y-fitting connected to the impeller housing for connection through two separate tubes to two fluid sources.

13. The system of claim 12 wherein the impeller housing includes a receptacle bonded thereto to which the Y-fitting is connected.

14. The system of claim 10 wherein the pinching device is a W-fitting connected to the impeller housing for connection to two separate tubes for connection to two fluid sources, the W-fitting being constructed and arranged so that the tubes are pinched if the pump device is removed from the support.

15. The system of claim 14 wherein the W-fitting includes a base member and opposed downwardly extending connectors for connection to the tubes.

16. The system of claim 1 wherein the support comprises a carriage including a micro switch that completes an electrical circuit to power the pump device when the carriage is suspended from an IV pole.

17. The system of claim 1 wherein the support comprises a hanging rod having an end connected to the pump device and forming a gravity switch to complete an electrical circuit to provide power to the pump device, the gravity switch being constructed and arranged so that the electrical circuit is completed only when the pump device is supported by the hanging rod.

18. The system of claim 1 wherein the support is a hanging sling.

19. The system of claim 3 further including a slide valve located in the impeller housing for connection with a fluid bag, the slide valve being open only when the pump device is supported in the support.

20. The system of claim 19 wherein the slide valve is slidably connected to the impeller housing so that if the flow detector is removed from the support it will drop under its own weight and slide relative to the slide valve so that the valve closes and prevents fluid flow.

21. The system of claim 1 wherein the fluid source is suspended from a carriage so that a gap exists between the arm of the pole and the top of the fluid bag, the gap preventing the pump from spiking to the fluid bag while hanging.

22. The system of claim 1 wherein the support comprises a bracket.

23. The system of claim 22 wherein the support comprises an L-bracket.
